(19) United States
(12) Patent Application Publication
(10) Pub. No.: US 2024/0268802 A1
(43) Pub. Date: Aug. 15, 2024

(54) CATHETER STATUS INDICATOR AND METHODS OF USE

(71) Applicant: PAVmed, Inc., New York, NY (US)

(72) Inventors: Michael Boutillette, New York, NY (US); Lishan Aklog, New York, NY (US); Brian J. deGuzman, New York, NY (US); Richard Yazbeck, New York, NY (US); Stephen J. O'Neill, New York, NY (US); Amos G. Cruz, New York, NY (US); Julian Fricks, New York, NY (US)

(21) Appl. No.: 18/290,889

(22) PCT Filed: Jul. 26, 2022
(86) PCT No.: PCT/US22/74148

§ 371 Date: Jan. 22, 2024

Related U.S. Application Data
(60) Provisional application No. 63/203,601, filed on Jul. 27, 2021.

(51) Int. Cl. A61B 10/04; A61B 10/02

(52) U.S. Cl. CPC A61B 10/04; A61B 2010/0216

(57) ABSTRACT

A device for collecting a biological sample in a patient, the device includes an expandable member sized for placement within the patient, the expandable member designed to transition between a plurality of states including a collapsed state and a sampling state; a handle in fluid communication with the expandable member; and an indicator configured to identify a status of the expandable member in response to the expandable member transitioning from one state to another.

### CROSS-REFERENCE TO RELATED APPLICATION

This application claims priority to, and the benefit of, U.S. Provisional Application No. 63/203,601, filed Jul. 27, 2021, for all subject matter common to both applications. The disclosure of said provisional application is hereby incorporated by reference in its entirety.

### FIELD OF THE DISCLOSURE

The present disclosure relates to a catheter device suitable for use within a body lumen. In particular, the present disclosure relates to a unique catheter construction for use in navigating a body lumen or vessel, such as a vein, artery, nasal cavity, esophagus, etc. to assist in the performance of various procedures, such as the collection of biological samples, and status indicators relating to same.

### BACKGROUND

A tissue collection device may include an expandable device. The device expands radially at a collection site within a body lumen, such as an esophagus. After the device is expanded, tissue is collected from the collection site. The expandable device is deflated after tissue is collected. Collected tissue is trapped inside the device when it is deflated after collection of the tissue. The tissue collection device may be inserted through an endoscope to the collection site or via standard catheter intubation techniques.

It is often difficult to ascertain the status of certain components of the device (e.g., the balloon) within the body. Specifically, such procedures may be performed without imaging in which case the operator may be uncertain that the expandable device has sufficiently inflated and/or deflated to properly collect and protect a sample from the patient's body.

The present disclosure is directed, in general, to a device for collecting a biological sample, and more specifically, to a device for collecting a biological sample, such as tissue, cells, protein, RNA and/or DNA from an esophagus of a patient.

### SUMMARY

In an embodiment, a device for collecting a biological sample in a patient is disclosed. The device includes an expandable member sized for placement within the patient, the expandable member designed to transition between a plurality of states including a collapsed state and a sampling state; a handle in fluid communication with the expandable member; and an indicator configured to identify a status of the expandable member in response to the expandable member transitioning from one state to another.

In some embodiments, the indicator can be an immovable indicator. The indicator can be a visual indicator that includes a plurality of icons, each of the plurality of icons corresponding to one of the plurality of states. The indicator can further include a processor configured and arranged to activate one of the plurality of icons based on a pressure within the device. The indicator can further include a memory to record at least one of pressure data and volumetric data during a procedure. The processor can be configured and arranged to differentiate between inflation and deflation of the expandable member. The processor can be configured and arranged to activate an icon associated with the sampling state only when the expandable member reaches a predetermined volumetric or pressure range as the expandable member is being deflated. The processor can be configured to activate an icon associated with the sampling state only when the expandable member reaches a predetermined volumetric or pressure range and stays within a range for a duration of more than 3 seconds.

In some embodiments, the immovable indicator can include three icons. In some embodiments, the immovable indicator can include four icons. The immovable indicator can include a plurality of icons arranged in a circle. The device can include an audible indicator corresponding to the immovable indicator. The expandable member can have a first volume in the sampling state and an indexing condition that corresponds to inflating the expandable member to a second volume, where the second volume can be greater than the first volume.

In an embodiment, a system for collecting a biological sample in a patient is disclosed. The system includes an expandable member, sized for placement within the patient, the expandable member having a plurality of sizes in a plurality of states, and a tube extending from the expandable member; a pressure source fluidly connected to the tube and configured to provide a pressure to the expandable member to actuate the expandable member to the plurality of sizes; a handle coupled to the tube; and an indicator to identify a size of the plurality of sizes of the expandable member.

In some embodiments, the plurality of sizes can include a collapsed state, a sampling state, and an indexing state, and the expandable member having a different size and shape in each of the plurality of sizes. The indicator can be an electronic visual indicator that includes a collapsed indicator, a sampling indicator, and an indexing indicator.

In an embodiment, a method for operating a catheter device is disclosed. The method includes providing a catheter device for insertion into a lumen, the catheter device including an expandable member having a plurality of states including a collapsed state and a sampling state, a handle fluidly coupled to the catheter device, and an indicator, the indicator being independent from the handle; performing a procedure by pressurizing or depressurizing the expandable member; and identifying a status of one of the plurality of states via the indicator.

In some embodiments, the method can further include the step of differentiating between inflation and deflation of the expandable member, and activating an icon associated with the sampling state only when the expandable member reaches a predetermined volumetric or pressure range as the expandable member is being deflated. The method can further include the step of recording at least one of volumetric data or pressure data during the procedure.

### BRIEF DESCRIPTION OF THE FIGURES

These and other characteristics of the present disclosure will be more fully understood by reference to the following detailed description in conjunction with the attached drawings, in which:

FIG. 1 is a cross-sectional view of a catheter device in accordance with the present disclosure;

FIG. 2 is an isometric view of a biological sample collection device constructed in accordance with the present disclosure;

FIG. 3 is an isometric view of the collection device of FIG. 2 shown in a collapsed configuration;

FIG. 4 is a cross-sectional view of the collection device of FIG. 3;

FIGS. 5A and 5B illustrate various examples of handles of a collection device;

FIGS. 6A, 6B, and 6C illustrate various examples of visual indicators on handles of collection devices;

FIGS. 7A, 7B, and 7C illustrate another example of a visual indicator using a proximal shaft with variable thicknesses;

FIGS. 8A-8D illustrate another example of a visual indicator using frosted lens;

FIGS. 9A-9C illustrate another example of a visual indicator using a moveable plunger; and

FIGS. 10A-10D illustrate the process of using a collection device within a patient.

### DETAILED DESCRIPTION

As used herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Like parts are designated by like reference numerals throughout, illustrate an example embodiment or embodiments of improved operation for the catheter device, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

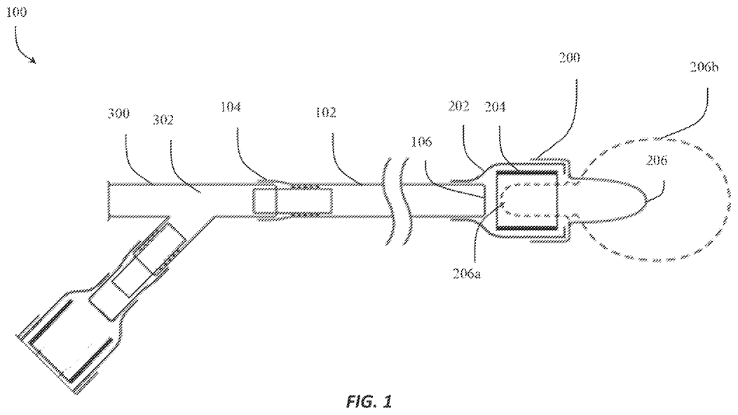

FIG. 1 depicts a cross-sectional side view of an exemplary catheter device 100 in accordance with the present disclosure. The catheter device 100 can include a procedural tool 200 at a distal section of the device and a connector 300 at a proximal section of the device 100, with a tubular member 102 extending therebetween. The procedural tool 200 and the connector 300 can include any combination of working ends for a catheter system or other procedural device that makes use of a tubular member 102. The procedural tool 200 and the connector 300 can be coupled to the tubular member 102 using any combination of mechanisms. For example, procedural tool 200 and the connector 300 can attach to the tubular member 102 via friction fit, adhesive, coupling mechanism, etc.

The tubular member 102 can be constructed from any combination of materials known in the art. For example, the tubular member 102 can be made from silicone rubber, nylon, polyurethane, polyethylene terephthalate (PET), latex, thermoplastic elastomers, etc., or a combination thereof. In some embodiments, the tubular member can be constructed to include a combination of other materials for desired structural effects. For example, the tubular member 102 can include braiding, reflow of polymers, etc. within the walls to achieve desirable properties (pushability, torquability, trackability, etc.), such that the tubular member has sufficient axial, or column, strength, and the ability to flex through tortious pathways or lumens.

In some embodiments, the tubular member 102 can include an open-ended channel extending from a proximal end 104 to a distal end 106. The tubular member 102 can also be any combination of tube-shaped devices, such as for example, a catheter. The tubular member 102 can also include any other combination of shapes without departing from the scope of the present disclosure, for example, the tubular member 102 can have cross-sections that are rectangular, ovular, polygonal, etc. In some embodiments, the proximal end 104 of the tubular member 102 can be in communication with the connector 300 of the device 100, while the distal end 106 of the tubular member 102 can be in communication with the procedural tool 200 of the device 100. As would be appreciated by one skilled in the art, the connector 300 and the procedural tool 200 can be coupled to the tubular member 102 in any combination of methods known in the art. For example, the procedural tool 200 and the connector 300 can be coupled to the tubular member 102 via a friction fit, a mechanical coupling, welding, thermal bonding, an adhesive, or combination thereof. The tubular member 102 can also be coupled to the procedural tool 200 and the connector 300 using different coupling systems or methods, alone or in any combination.

In some embodiments, the procedural tool 200 can be coupled to the distal end 106 of the tubular member 102. The procedural tool 200 can include any combination of elements to both couple to the tubular member 102 and perform a procedure with the assistance of the tubular member 102. For example, the procedural tool 200 can include a procedural housing 202 (e.g., a capsule), a stiffening sleeve 204, and/or a working end 206 (e.g., one or more balloons). The procedural housing 202 can be coupled to or be an extension of the distal end 106 of the tubular member 102. For example, the procedural housing 202 can be coupled to the outside or inside of the tubular member 102 or can be an extension thereof. In some embodiments, the tubular member 102 and/or procedural housing 202 can be constructed from a substantially soft material to provide comfort to a patient. For example, the procedural housing 202 can be constructed from a polypropylene material to assist in swallowing the device 100. The procedural housing 202 can be a solid structure, it can include a hollow cavity, it can include a mechanical tool, or a combination thereof that with access to the channel of the tubular member 102 via an open end at the distal end 106. As would be appreciated by one skilled in the art, the catheter device 100 can include any combination of procedural tools known in the art coupled to the procedural tool 200 without departing from the scope of the present disclosure.

In some embodiments, a stiffening sleeve 204 can be included within a cavity of the procedural housing 202 to provide a sufficiently rigid structure to provide hoop strength and for maintaining or creating a desired shape of the procedural housing 202. The stiffening sleeve 204 can be positioned at the distal end of the tubular member 102 to provide sufficient stiffness thereto so that a removable working end 206 can be attached to the tubular member 102. The stiffening sleeve 204 can be any combination of shapes and sizes to provide strength to the procedural housing 202. For example, the stiffening sleeve 204 can be a substantially tubular shaped device wrapping around at least a portion of the inner circumference of the procedural housing 202. Similarly, the stiffening sleeve 204 can be constructed from any sufficiently rigid materials, such as for example, metal, plastic, etc. to provide a sufficiently rigid structure of the housing 202. The stiffening sleeve 204 can be positioned within the procedural housing 202 through any combination of means known in the art. For example, the stiffening sleeve 204 can be installed within the procedural housing 202 via a friction fit, injection molding, etc. In some embodiments, the stiffening sleeve 204 can be provided at the end of the tubular member 102 without the presence of a procedural housing 202.

In some embodiments, as depicted in FIG. 1, the working end 206 can be configured with one or more expandable members, e.g., balloons, to expand a location, anchor part of the working end 206, contact an inner wall of the body, deliver medication, etc. The one or more balloons can be coupled to the procedural housing 202, for example, at an end opposing the end of the procedural housing 202 coupled to the tubular member 102. The working end 206 can be coupled to or be an extension of the procedural housing 202. For example, the working end 206 can be coupled to the outside or inside of the housing 202 structure, the tubular member 102, or a combination thereof. In some embodiments, the working end 206 can be constructed from a substantially flexible material to provide a transition between states (e.g., inflated or deflated) when a positive or negative pressure is applied through the tubular member 102. For example, the working end 206 can be constructed from a rubber or silicone material. In some embodiments, the tubular member 102, the procedural housing 202 and the working end 206 can all be constructed from the same material, for example, silicone.

In some embodiments, the working end 206 can be configured to transition between a plurality of states depending on a level and type of pressurization applied through the tubular member 102, via the procedural housing 202 cavity. The working end 206 can be configured to transaction back and forth between the different states 206, 206a, 206b, as well as other intermediate states that are smaller or larger than states 206a, 206b. For example, a positive pressure can be applied via the connector 300 and through the tubular member 102 to inflate the working end 206 (to state 206b) and then a negative pressure can be applied to deflate the working end 206 and optionally retract the working end 206 into the procedural housing 202 (to state 206a). As would be appreciated by one skilled in the art, depending on the configuration of the working end 206, the application of positive or negative pressure can have effects not limited to inflation/deflation of one or more balloons. For example, changes in pressurization applied to the working end 206 can cause components of the working end to actuate, open/close, rotate, etc.

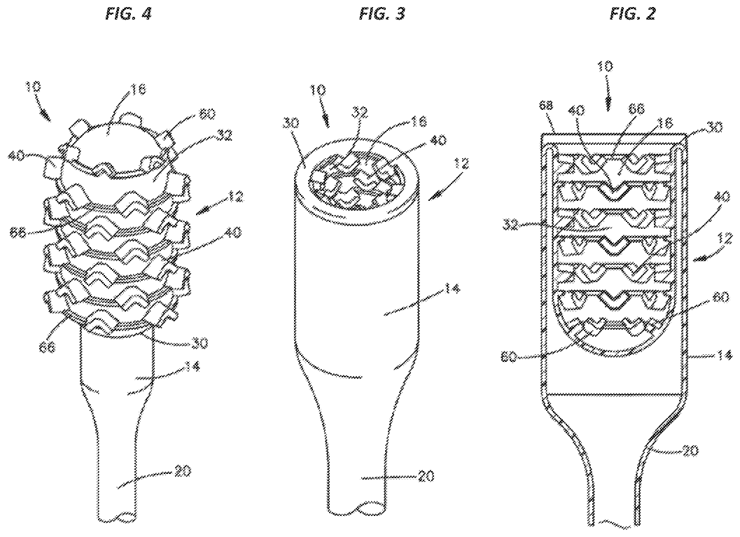

Several examples of procedural tool 200 are possible, including those that include a balloon-type working end 206 and a capsule-type procedural housing 202. In some examples, the procedural tool 200 is a cell collection device similar to that described in U.S. Pat. No. 10,660,621 to Markowitz et al., the disclosure of which is hereby incorporated by reference in its entirety as if fully set forth herein. By way of illustration, procedural tool 200 may include a collection device 10 for the collecting a biological sample constructed in accordance with the present disclosure as illustrated in FIGS. 2-4. The collection device 10 may be used to collect tissue, cells, protein, RNA and/or DNA from a body lumen, such as an esophagus of a patient, and the tissue, cells, protein, RNA and/or DNA collected from the esophagus may be used in any one of the methods disclosed in U.S. patent application Ser. No. 14/109,041, U.S. patent application Ser. No. 13/670,155, U.S. patent application Ser. No. 13/263,020, U.S. Pat. Nos. 8,642,271, 8,481,707, 8,415,100, 8,221,977, 7,964,353, and 7,485,420, which are incorporated therein by reference in their entirety.

As shown in FIG. 2, collection device 10 can include a generally hollow longitudinally extending collection portion 12. The collection portion 12 can have a first or proximal axial end portion 14 and a second or distal axial end portion 16. The proximal axial end portion 14 and the distal axial end portion 16 may be made of a flexible polymer, such as silicone or polyurethane. The distal axial end portion 16 can have a lower durometer than the proximal axial end portion 14. The distal axial end portion 16 may expand and contract. The first or proximal axial end portion 14 can be relatively rigid (i.e., more rigid than the distal axial end portion 16). Therefore, the proximal end portion 14 can have a fixed radial extent. The first axial end portion 14 and the second axial end portion 16 may be integrally formed as one-piece or may be formed as separate pieces that are connected together in any desired manner. Although the proximal end portion 14 is illustrated as having a cylindrical shape, the proximal end portion may have any desired shape.

The proximal axial end portion 14 can be connected to a support member 20, such as a catheter. The support member 20 may be a tubular member in fluid communication with the interior of the collection portion 12. The proximal axial end portion 14 can conduct a fluid, such as air, from the support member 20 to the distal axial end portion 16. A proximal end of the support 20 may be connected to a stopcock and syringe, or other inflating device, to control the injection of fluid to move the distal end portion 16 between the collapsed and expanded positions. The support 20 may resist collapsing when a vacuum is applied to the support member and resists stretching during withdrawal of the collection device 10 from the collection site.

The second or distal end portion 16 of the collection portion 12 may have an expanded, or inflated, position (FIG. 2), and a collapsed, or deflated, position (FIGS. 3-4). The expanded position shown in FIG. 2 may be one of many expanded positions for the distal end portion 16. It is contemplated that the distal end portion 16 may expand more than shown in FIG. 2 so that the distal end portion obtains a more spherical shape and looks similar to a hot air balloon to be used, for example, in indexing or locating a feature within the body, for example similar to state 206b of FIG. 1. The distal end portion 16 can have a convex shape, as shown in FIG. 2, when in the expanded or inflated position. The distal end portion 16 may extend radially outward a greater distance than the proximal end portion 14 when in the expanded position.

The distal end portion 16 can extend proximally into the first or proximal axial end portion 14. The distal end portion can have a concave shape, shown in FIGS. 3 and 4, when in the collapsed or deflated position. The distal end portion 16 may be inverted when in the collapsed position. The distal end portion 16 can extend axially into the interior of the proximal end portion 14 when in the collapsed or deflated position, as seen in FIG. 4. Therefore, the distal end portion 16 moves axially or longitudinally relative to the proximal end portion 14 when moving between the deflated and inflated positions. The distal end portion 16 may be biased into the collapsed or deflated position in any desired manner, for example by the application of a fluid pressure or vacuum.

The proximal end portion 14 can have a relatively high durometer so that the proximal end portion does not collapse when a vacuum is applied to the proximal end portion through the support 20. In some embodiments, the shape of the proximal end portion 14 does not change when the distal end portion 16 moves between the deflated and inflated positions. In some embodiments, the proximal end portion 14 does not move radially when the distal end portion 16 moves between the deflated and inflated positions.

The distal end portion 16 of the collection portion 12 may be connected to the proximal end portion 14 by a circumferentially extending hinge 30. The hinge 30 may be a fold of material. The hinge 30 may extend circumferentially around the collection portion 12. The hinge 30 can define a distal axial end surface of the collection portion 12 when the distal end portion 16 is in the deflated or collapsed position. The distal end portion 16 can define an axial end surface of the collection portion 12 when the distal end portion is in the inflated or expanded position.

The distal end portion 16 has an outer surface 32 for collecting tissue when the distal portion is in the expanded position. The outer surface 32 faces radially outwardly when the distal end portion 16 is in the expanded position and may face radially inwardly when the distal end portion is in the collapsed or inverted position. It is contemplated that the outer surface 32 of the distal end portion 16 may have any desired construction for collecting tissue. For example, the outer surface 32 of the distal end portion 16 may have a plurality of projections or bristles 40 for collecting tissue. The distal end portion 16 may have any desired number of projections or bristles 40. The projections or bristles 40 may have a V-shape but other shapes are possible. In some examples, the distal end portion 16 may include a plurality of projections or bristles 60 (FIG. 2) extending from a distal portion of the distal end portion 16. The projections 60 have the same general shape as the projections 40 and may be of a same size or smaller than the projections 40. The projections or bristles 40, 60 may be arranged in circumferentially extending rows (FIG. 2). It is contemplated that each row has six projections 40 or 60. It is contemplated that each of the rows may have any desired number of projections 40 or 60. Each of the projections 40, 60 may be circumferentially offset from the projections on an adjacent row. Ribs 66 can extend circumferentially between adjacent projections 40, 60 in each row.

The collection portion 12 can be moved to a collection site within a body lumen, such as an esophagus, with the distal end portion 16 in the collapsed or deflated position. The collection portion 12 may be swallowed by a patient. It is also contemplated that the patient may be intubated with the collection portion 12 attached to a catheter. The distal end portion 16 may be held in the collapsed or deflated position by the hinge 30 and/or by applying a vacuum to the collection portion 12 through the support 20. The distal end portion 16 may also be held in the collapsed position by a cap 68 or cover that falls off, pops off and/or dissolves when the collection portion 12 reaches the body lumen. The support member 20 or catheter may have depth markings to determine the collection site within the patient's anatomy. The distal end portion 16 can be moved from the collapsed position (FIGS. 3 and 4) to the expanded position (FIG. 2) when the collection portion 12 is at the collection site. A pressurized fluid, such as air, may be applied to the distal end portion 16 to cause the distal end portion to move axially from the collapsed position to the expanded position.

The collection portion 12 can be moved in the body lumen to collect a biological sample, such as, tissue, cells, protein, RNA and/or DNA from the collection site when the distal portion is in the expanded position. It is contemplated that the collection portion 12 may only be moved in a proximal direction so that the expanded distal end portion 16 can engage the collection site to collect biological samples. Depth markings on the support member 20 or catheter may be used as a guide. After the biological sample is collected, the distal end portion 16 can be moved from the expanded position to the collapsed or inverted position. The distal end portion 16 may be moved from the expanded position to the collapsed position by applying a vacuum to the collection portion 12. As the collection portion 12 moves out of the body lumen, in some examples, the distal end portion 16 does not engage the body lumen and prevents the collected biological samples from being contaminated by tissue from areas along the body lumen different from the collection site. Once the collection device 10 is removed from the patient, the biological samples can be collected via a wash and/or the collection portion 12 or the distal end portion 16 may be cut from the support member 20 and deposited in a biological sample vial.

Continuing with FIG. 1, in some embodiments, the connector 300 of the device 100 can be include or otherwise be attached to an input mechanism for pressurizing and depressurizing the tubular member 102. The pressurization or depressurization of the tubular member 102 can be provided for controlling the states of the working end 206 at the procedural tool 200. For example, the connector 300 of the device can include a Y-connector 302 for accepting a handle (not shown) having a mechanism (e.g., a syringe or other pressure-producing element) for controlling the state of the working end 206. As would be appreciated by one skilled in the art, the functionality of the Y-connector 302 can be implemented using another connection, such as for example, a T-connector or V-connector. The Y-connector 302 (or other connector) can be used to introduce gas or fluid into the tubular member 102 to modify the state of the working end 206. For example, the Y-connector 302 can receive gas or fluid from a syringe or other input and pass the gas or fluid through the channel of the tubular member 102 into the cavity of the procedural housing 202. Similarly, the Y-connector 302 can be used to withdraw gas or fluid from the tubular member 102 or the cavity of the procedural housing 202.

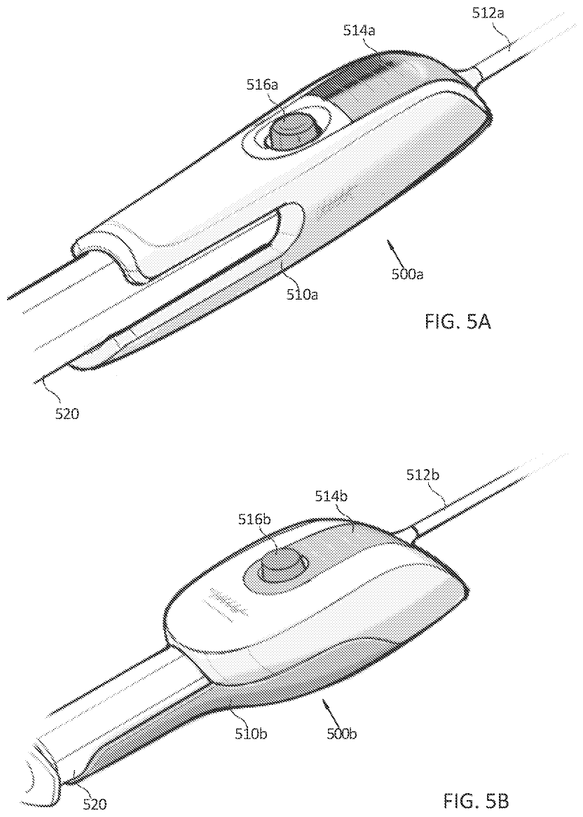

A handle for accommodating a syringe or other inflating components may be used to transition the cell collection device between the various conditions in addition to, or in place of the connector 300. For example, a handle may be connected upstream or downstream of the connector 300. Alternatively, the functionality, and structure, of the connector 300 can be incorporated into the handle. The handle can be ergonomically designed to provide the user with sufficient comfort and control during use of the device 100. FIGS. 5A and 5B illustrate two examples of such handles.

As illustrated in FIG. 5A, a handle 500a can generally include an elongated body 510a connected to a shaft 512a having a lumen. The handle can be configured to receive at least a portion of a syringe 520 and modulate the delivery of a fluid from the syringe to a shaft 512a. In some embodiments, the shaft 512a can be connected at a distal end to a collection device, e.g., collection device 10. To that end, handle 500a may include a stopcock 516a for regulating the fluid flow to a collection device. One or more indicators 514a may be disposed on the handle 500a, such that the user of the device is alerted, or notified, of the status of a collection device. In some embodiments, the indicator 514a can be a separate module from the handle 500a. The indicator 514a, or any of the below disclosed indicators, can be arranged as independent modules from the respective handles. In some examples, the indicator 514a can include lamps, lightpipes, backlight lighting, backlight lenses, membranes, e-ink, and/or displays (e.g., LCD, LED, etc.), or combinations thereof. The indicator 514a can include any number of input control signals, including pressure sensors configured to determine the internal pressure within the lumen of the shaft 512a. In the case of pressure sensors, the handle 500a can be configured such that when the pressure reaches a given range, a controller can understand that the collection device is arranged in a given state, e.g., collapsed or expanded. The controller can then convert the input pressure signal to the indicator 514a to notify the user. Other input control signals can include other mechanical, electrical, optical sensors that are configured to determine the relative shape and size of a collection device disposed at the distal end of the shaft 512a.

FIG. 5B illustrates a second example of a handle 500b. Handle 500b can includes a relatively wider body 510b that can be connected to a shaft 512b having a lumen. The handle can be configured to receive at least a portion of a syringe 520 and modulate the delivery of a fluid from the syringe to the shaft 512b and into a collection device. In a similar manner, handle 500b may include a stopcock 516b for regulating the fluid flow. Similar to handle 500a, handle 500b can include one or more indicators 514b to provide a user with an active indicator of the status of a collection device disposed within a lumen of a patient.

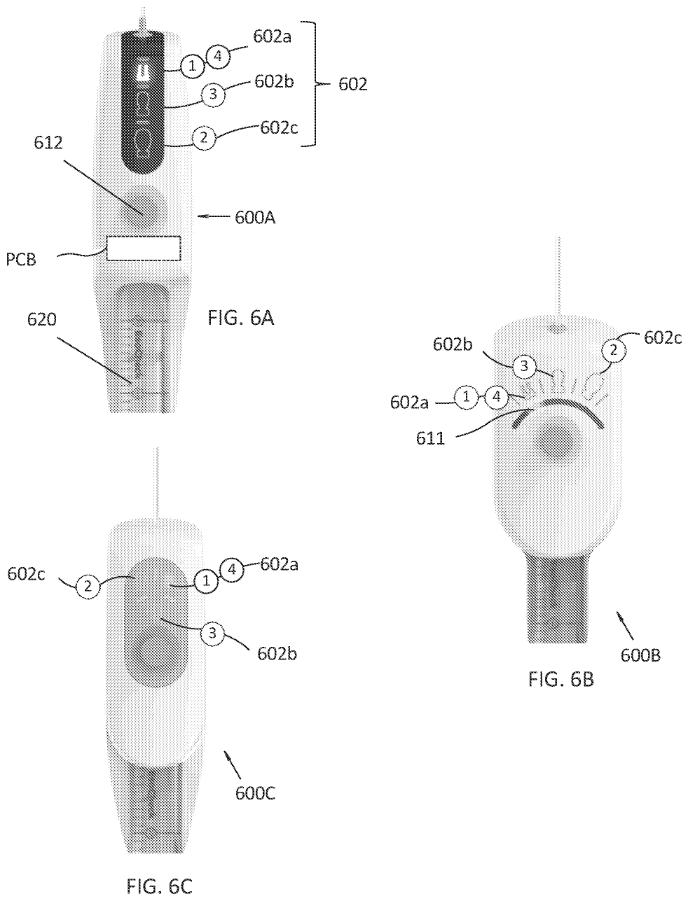

FIGS. 6A, 6B, and 6C illustrate certain examples of handles having visual, immovable, indicators. In a first example, as shown in FIG. 6A, an elongated handle 600A includes a set of indicators 602 arranged as a vertical series of illuminating icons 602a, 602b, 602c, disposed, for example, adjacent stopcock 612. Though the indicators 602 are shown as being visual illuminating icons 602a-c, it will be understood that audible indicators (e.g., beeps, chimes, etc.) and/or tactile indicators may be used in addition to, or instead of, the visual indicators. Alternatively, other indicators are considered to be within the scope of this disclosure, including those that are movable. Each of the illuminating icons 602a-c may correspond to one or more of the collection device states described above with respect to FIGS. 1-4. For example, a first, illuminating, icon 602a may represent the collection device in the collapsed, inverted condition and may graphically indicate the state via a drawing or silhouette of the collection device in that state. A second, illuminating, icon 602b may represent the collection device in a partially-inflated sampling condition. A third icon 602c may represent the collection device in an inflated, indexing, condition. The indicators 602 may sequentially illuminate in a predetermined order during the procedure to guide the operator. For example, the first icon 602a indicating the collapsed condition may initially be illuminated or otherwise marked to show that the collection device is properly inverted and collapsed and ready for delivery to a site of interest. As the user inflates the collection device via a syringe 620 or other mechanism, the first icon 602a may dim, turn off, or otherwise be toggled off in some manner, and another icon 602b, 602c may illuminate in due course. In some examples, the sampling condition icon 602b can initially be skipped and the third icon 602c can be illuminated once the indexing condition is reached. In some examples, where the device is being used to collect a sample from the esophagus, after the user locates the gastroesophageal junction and begins to slowly deflate the collection device to achieve the sampling condition, the third icon 602c may be turned off and the second icon 602b may be activated or triggered. After proper sampling, the operator may deflate the collection device with the stopcock 612 to bring the collection device to the collapsed or inverted condition and the first icon 602a may be activated to alert the user that it is safe to remove the device from the lumen of the patient.

By way of illustration, the order of the icon activation is shown for each icon by a circled numeral. In at least some examples, only a single icon of the plurality of icons is illuminated at any moment as a function of the volume of fluid moved from the syringe 620 to the collection device, or as a function of the internal pressure, or other variables. Additionally, or alternatively, a blinking or odd-colored icon (e.g., red) may indicate an error state to alert the user that a possible leak is detected. In one variation, the sampling condition icon 602b may be triggered when moving from the collapsed condition to the indexing condition. In another variation, to avoid confusion during inflation the sampling condition icon 602b can be skipped if a specific volume (e.g., between 5 and 6 cc) of the balloon is reached for only a short transitory duration (e.g., for less than 10 seconds or less than 5 seconds), and the indexing condition icon illuminates after the collapsed condition upon inflation. The sampling condition icon 602b may also be activated only when the system senses that the volume inside the balloon decreases from a first volume that is larger than the sampling volume, which corresponds to the change from the indexing condition to the sampling condition.

The indicators 602 (e.g., the illuminated icons 602a-c) may be controlled by electronics disposed within the handle. These electronics may be in the form of a printed circuit board “PCB” and include a processor, a memory, a battery and/or a telemetry unit (e.g., a transceiver). In at least some examples, the processor is configured to continuously measure and record a volume of the balloon and/or a pressure in the shaft throughout the procedure. Alternatively, or additionally, the processor can be configured to measure other variables, conditions, or states, of the system to provide an indication of the status. The measured volumes and/or pressures may be stored on a memory within the handle (or transmitted wirelessly to external servers via the telemetry unit) so that the device can be continuously monitored. In some examples, by continuously monitoring and storing the volumes within the collection device and/or a pressure in the shaft, it is possible to track and troubleshoot failure within a balloon or other collection device (e.g., to know when a leak may occur in a procedure or at what volume an error typically occurs). The processor may also control which light is to be activated or illuminated during the procedure. The processor may also signal to the operator that they should perform an action (i.e., inflate, deflate, remove, etc.).

Another example of a graphical indicator is shown in FIG. 6B, which shows a speedometer-type of visualization 611. In this example, handle 600B can includes a first icon 602a representing the collection device in a collapsed, inverted condition, a second icon 602b representing the collection device in a partially-inflated sampling condition, and a third icon 602c representing the collection device in an inflated indexing condition. In addition, handle 600B can include a curved lightpipe 611 underneath each of these icons. Alternatively, the lightpipe visualization 611 can have any linear, or non-linear, shape. In this example, the lightpipe 611 may illuminate underneath each of the icons 602a-c from one side to the other beginning with a first icon 602a, followed by the indexing condition icon 602c, then the sampling condition icon 602b, and then the collapsed condition icon 602a, the steps being activated at the appropriate time throughout the procedure based on the sensed volume of the collection device and/or a pressure in the shaft.

In a third example, illustrated in FIG. 6C, a circular visualization arrangement is shown where handle 600C includes a first icon 602a representing the collection device in collapsed, inverted condition, a second icon 602b representing the collection device in a partially-inflated sampling condition, and a third icon 602c representing the collection device in an inflated indexing condition. While a circular visualization arrangement is illustrated, other shapes can be used. In this example, the icons 602a-c may light up beginning with the first icon 602a, then proceed counter-clockwise with the indexing condition icon 602c, then the sampling condition icon 602b, and then complete the circle by returning to the collapsed condition icon 602a, the steps being activated at the appropriate time throughout the procedure based on the sensed volume of the balloon and/or a pressure in the shaft.

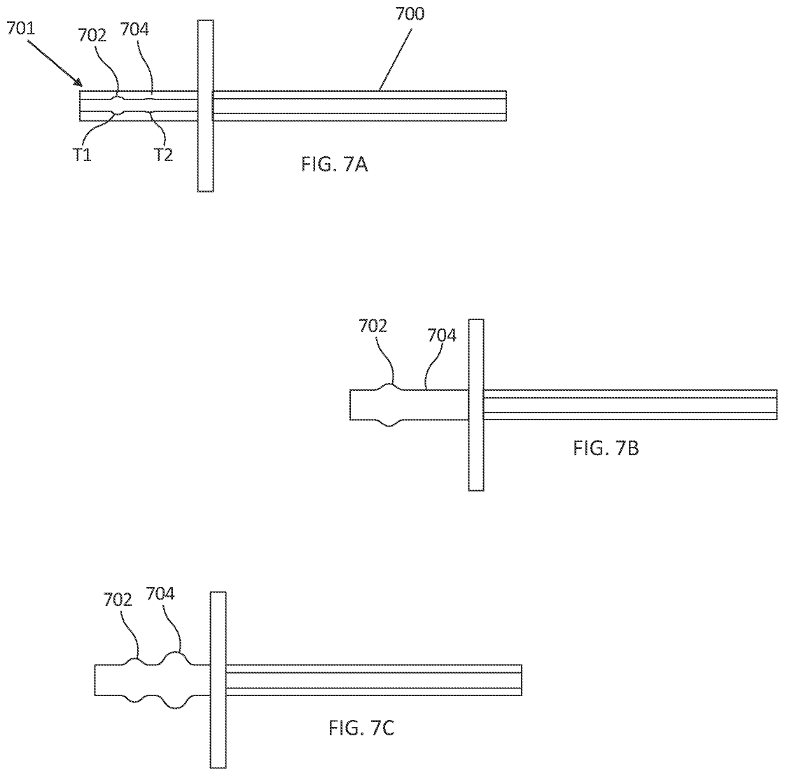

Other indicator types are also possible. For example, FIGS. 7A, 7B, and 7C illustrate a movable visual indicator in the form of a proximal end expanding shaft 700. A proximal portion 701 of the shaft 700 can be in fluid communication with an expandable collection device and may have a plurality of sections of walls with different thicknesses T1, T2 that will visually, or tactically, alert the operator of the interior pressures. The wall thicknesses T1, T2 can extend circumferentially about the entire circumference of the expanding shaft 700. Thicknesses T1, T2 may correspond to medium and high pressures, corresponding to, for example, the sampling condition and the indexing condition of the collection device, respectively. In this example, proximal portions 701 of shaft 700 may have a first radial section(s) 702 with the thinnest wall thickness T1 that can expand, or bulge out, under a medium pressure of the sampling condition, as shown in FIG. 7B. A second section(s) 704 with may expand, or bulge out, under a moderate pressure, e.g., under the high pressure of the indexing condition as seen in FIG. 7C). The second section 704 can have a second wall thickness T2 that is thicker than the wall thickness T1 of proximal portions 701, but larger than remaining portions of the shaft. Thus, the approximate pressure within the shaft, and the corresponding collection device volume, may be noted through visual observation of the status of the proximal expanding shaft and particularly by seeing whether one or more portions of the shaft have created a bulge. Alternatively, the user may feel the raised portions with their hand and be notified of the status of the collection device without needing visual confirmation.

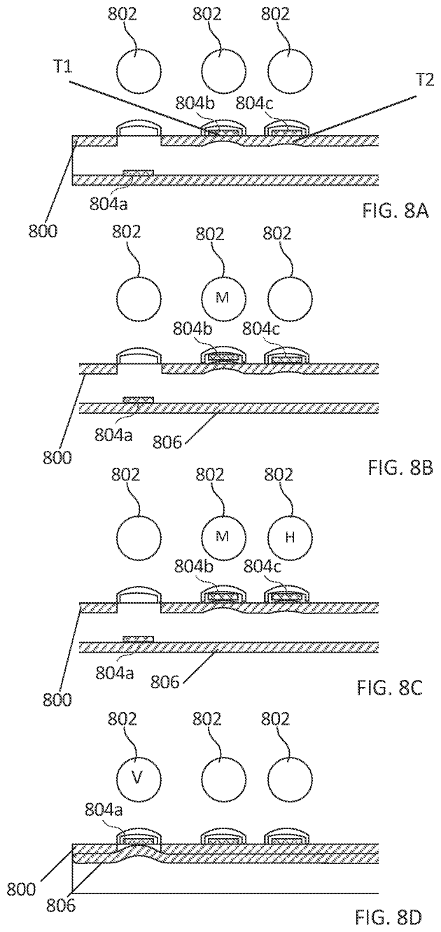

In another example, as shown in FIGS. 8A, 8B, 8C, and 8D, windows, e.g., frosted lenses 802 may be disposed over a portion of the shaft 800, and a plurality of buttons, e.g., raised buttons 804a, 804b, 804c having markings may be disposed below the frosted lenses 802. The shaft 800 may have variable thicknesses T1, T2 under a portion of the frosted lenses so that raised buttons 804b and 804c are pushed up toward the frosted lenses 802 and become visible at predetermined pressures, similar to the previous embodiment of FIGS. 7A-C. In each of FIGS. 8A-D, a cross-sectional portion of the shaft is illustrated as well as three visualization circles that would be seen from a top view. For example, as shown in FIG. 8A, in a first state no markings are shown through the frosted lenses 802, which are illustrated as three empty circles. The first state may represent the system at rest, with atmospheric pressure. In a second state, a predetermined moderate pressure may push the area having a first thickness T1 to actuate one of the raised buttons 804b and push it against the frosted lens so that an “M” for medium pressure is visible through the lens. This medium pressure may correspond to a sampling condition of the collection device. With additional pressure introduced into the shaft 800, the area having the second thickness T2 can deform upward such that the button 804c is pushed toward its corresponding lens 802 and the “H” for high pressure may also be seen to indicate the indexing condition of the collection device, as seen in FIG. 8C. In the presence of a vacuum pressure in the shaft 800, an internal shaft 806 can be drawn upward towards shaft 800 such that button 804a may be advanced towards and against lens 802 and a “V” may appear, as seen in FIG. 8D. It will be understood that instead of “M”, “H” and “V”, that the buttons may instead display symbols or completed words, such as, “SAMPLING,” “INDEXING,” and “VACUUM.”

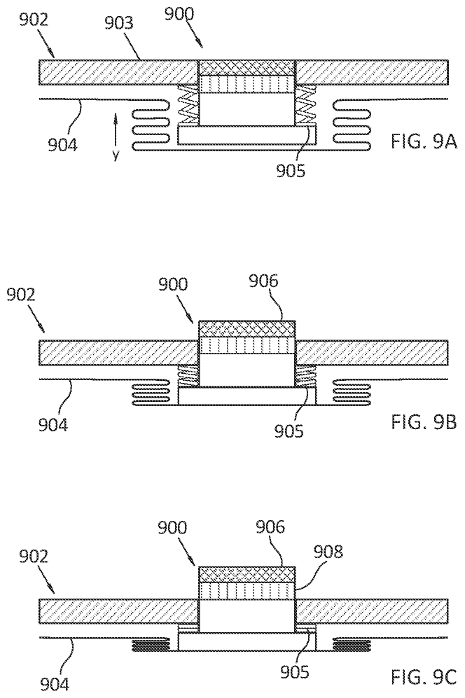

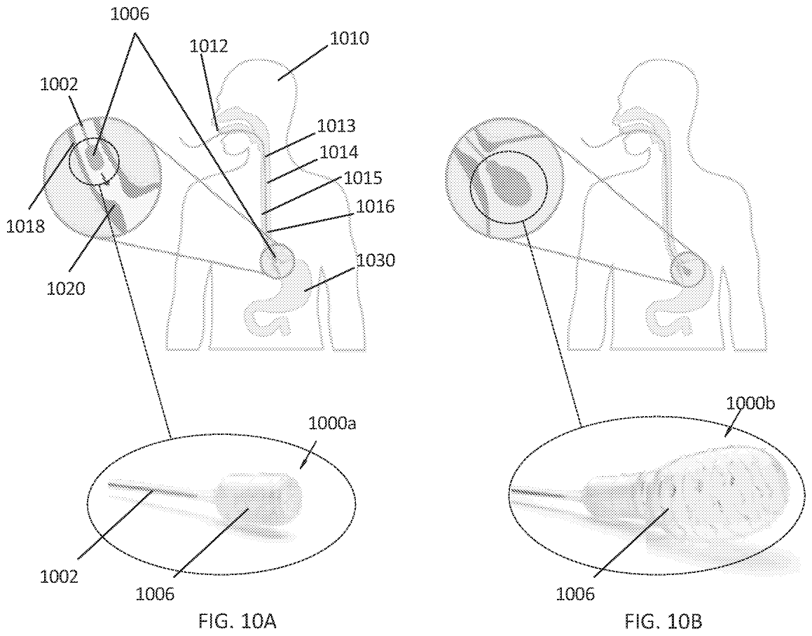

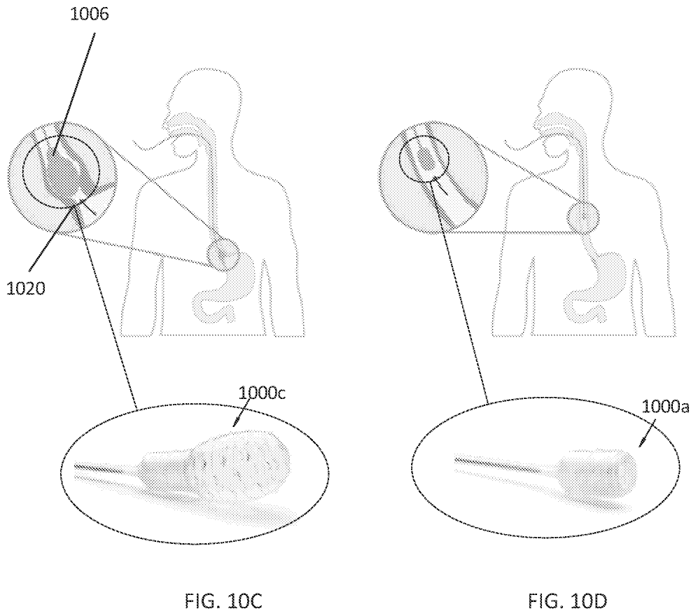

In yet another example, as shown in FIGS. 9A, 9B, and 9C, a spring, for example a spring-loaded plunger, may be used to indicate the status of the collection device within the patient's body. For example, a plunger 900, or piston, may be disposed adjacent shaft 902 and bounded by a seal. For example, the seal can be a bellows seal 904. The plunger 900 may be seated on one or more springs 905. A pressure inside shaft 902 can urge plunger 900 upward in the direction of arrow “Y” against the force of the spring so that a portion of the plunger 900 is visible. The visible portions 906, 908 of the plunger 900 can correspond to, and indicate, the pressure inside the shaft 902 and thus a condition of the collection device, as discussed above with respect to the prior embodiments. As illustrated in FIG. 9A, a plunger 900 can be arranged approximately flush with the outer surface 903 of the shaft 902, indicating that there is no pressure inside the shaft. In FIG. 10B, the shaft 902 can be inflated to a first pressure such that the bellows seal 904 advances the plunger 900 to a first visible portion 906, indicating a medium pressure inside the shaft, for example, corresponding to a sampling volume of the collection device. In FIG. 9C, the shaft 902 can be inflated to a second pressure that is greater than the first pressure, such that the bellows seal 904 advances the plunger 900 further and a second visible portion 908 is revealed that indicates a high pressure inside the shaft, for example, corresponding to an indexing volume of the balloon. Portions of the plunger 900 may be pattern-coded or color-coded to quickly identify the pressure within the shaft. For example, a plunger may have an upper green ring, a middle yellow ring, and a lower red ring. Each of the rings can correspond to zero pressure, sampling pressure and indexing pressure, respectively.

FIGS. 10A-D illustrate a use of a collection device within the esophagus for early detection of medical conditions of the esophagus, such as pre-cancerous conditions including, but not limited to, Barrett's Esophagus. Specifically, each of FIGS. 10A-D illustrate the position of a cell collection device 1006 within the body, and an approximate state of the cell collection device 1006 at that given position. Any of the handles disclosed above can be used with the cell collection device 1006 to indicate to the user the state of the cell collection device 1006. For example, the cell collection device 506 can be an expandable balloon. In FIG. 10A, the cell collection device 1006 can be disposed in its first, collapsed or inverted, condition 1000a, for easy swallowing by the patient 1010. The collapsed condition 1000a may be approximately the shape of a pill with the balloon being completely inverted and housed within the capsule, as shown in FIGS. 3 and 4, the cell collection device 1006 being coupled to a catheter 1002 via a support member as described with respect to FIGS. 2-4. The catheter 1002 may be advanced through the mouth 1012, past the cervical esophagus 1013, the upper thoracic esophagus 1014, the middle thoracic esophagus 1015 and down toward the lower thoracic esophagus 1016. Still in its collapsed state, the cell collection device may be advanced past the gastroesophageal junction (GEJ) 1018 toward the stomach 1030. Optionally, the cell collection device 1006 may be inflated to a second condition shown in FIG. 10B. This second condition may be referred to as the indexing condition 1000b, and it may include inflating the balloon of the cell collection device 1006 to a volume or size that is larger than the sampling condition 1000c. In at least some examples, the indexing condition 1000b can include delivering approximately 11 cc of fluid, e.g., air, to the cell collection device 1006 to bring it to this particular inflated state. The purpose of the second condition may be to identify the position of the gastroesophageal junction 1018 to ensure that cell collection is performed at the appropriate position within the esophagus. Specifically, by inflating the balloon to an indexing condition 1000b right outside the gastroesophageal junction 1018, a landmark may be identified. The operator may gently pull back on the catheter 1002 until a moderate resistance is felt as a result of the balloon coming up against the lower esophageal sphincter (LES) 1020. Once the lower esophageal sphincter 1020 is located, the operator may note the location of catheter markings at the dental arches.

The cell collection device may then be slightly deflated to its third condition, a sampling condition 1000c, which is slightly smaller than indexing condition 1000b. In at least some examples, the sampling condition 1000c can include inflating (or deflating to this condition if performed after the indexing condition) the cell collection device with approximately between 5 cc and 6 cc of fluid (e.g., 5.4 cc or 5.6 cc) of fluid. The handle can then indicate via visual, tactile, or auditory alert to the user the condition of the cell collection device. To sample the biological tissue, the operator may continue to gently pull the device in a retrograde direction across the LES 1020 into and up the esophagus to the desired distance in order to gather a sample from the targeted portion of the esophagus, as seen in FIG. 10C). In one example, the operator may sample a minimum distance of 5 cm of the distal esophagus from the LES before the cell collection device 1006 is deflated and retracted into the capsule to ensure capture of any possible abnormal columnar epithelial cells that could indicate, for example, Barrett's Esophagus (BE). After the desired length is sampled, as noted by monitoring the length markings on the catheter, the operator may invert the cell collection device 1006 with the collected esophageal cell samples into the capsule back to the collapsed condition 1000a, as seen in FIG. 10D, and pull back the catheter to retrieve it out of the patient. In order to maintain and not disturb the cellular material collected from the distal esophagus, the balloon can be deflated and inverted at a distance not greater than 15 cm from the LES.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the disclosure. It is intended that the present disclosure be limited only to the extent required by the appended claims and the applicable rules of law.

It is also to be understood that the following claims are to cover all generic and specific features of the disclosure described herein, and all statements of the scope of the disclosure which, as a matter of language, might be said to fall therebetween.

What is claimed is:

1. A device for collecting a biological sample in a patient, the device comprising:
an expandable member sized for placement within the patient, the expandable member designed to transition between a plurality of states including a collapsed state and a sampling state;
a handle in fluid communication with the expandable member; and
an indicator configured to identify a status of the expandable member in response to the expandable member transitioning from one state to another.

2. The device of claim 1, wherein the indicator is an immovable indicator.

3. The device of claim 2, wherein the indicator is a visual indicator that includes a plurality of icons, each of the plurality of icons corresponding to one of the plurality of states.

4. The device of claim 3, wherein the indicator further includes a processor configured and arranged to activate one of the plurality of icons based on a pressure within the device.

5. The device of claim 4, wherein the indicator further includes a memory to record at least one of pressure data and volumetric data during a procedure.

6. The device of claim 4, wherein the processor is configured and arranged to differentiate between inflation and deflation of the expandable member.

7. The device of claim 6, wherein the processor is configured and arranged to activate an icon associated with the sampling state only when the expandable member reaches a predetermined volumetric or pressure range as the expandable member is being deflated.

8. The device of claim 4, wherein the processor is configured to activate an icon associated with the sampling state only when the expandable member reaches a predetermined volumetric or pressure range and stays within a range for a duration of more than 3 seconds.

9. The device of claim 2, wherein the immovable indicator includes three icons.

10. The device of claim 2, wherein the immovable indicator includes four icons.

11. The device of claim 2, wherein the immovable indicator includes a plurality of icons arranged in a circle.

12. The device of claim 2, further comprising an audible indicator corresponding to the immovable indicator.

13. The device of claim 1, wherein the expandable member has a first volume in the sampling state.

14. The device of claim 13, further including an indexing condition that corresponds to inflating the expandable member to a second volume, the second volume being greater than the first volume.

15. A system for collecting a biological sample in a patient, the system comprising:
an expandable member, sized for placement within the patient, the expandable member having a plurality of sizes in a plurality of states, and a tube extending from the expandable member;
a pressure source fluidly connected to the tube and configured to provide a pressure to the expandable member to actuate the expandable member to the plurality of sizes;
a handle coupled to the tube; and
an indicator to identify a size of the plurality of sizes of the expandable member.

16. The system of claim 15,
the plurality of sizes include a collapsed state, a sampling state, and an indexing state, and
the expandable member having a different size and shape in each of the plurality of sizes.

17. The system of claim 15, wherein the indicator is an electronic visual indicator that includes a collapsed indicator, a sampling indicator, and an indexing indicator.

18. A method for operating a catheter device, the method comprising:
providing a catheter device for insertion into a lumen, the catheter device including an expandable member having a plurality of states including a collapsed state and a sampling state, a handle fluidly coupled to the catheter device, and an indicator, the indicator being independent from the handle;
performing a procedure by pressurizing or depressurizing the expandable member; and
identifying a status of one of the plurality of states via the indicator.

19. The method of claim 18, further comprising the step of differentiating between inflation and deflation of the expandable member, and activating an icon associated with the sampling state only when the expandable member reaches a predetermined volumetric or pressure range as the expandable member is being deflated.

20. The method of claim 18, further comprising the step of recording at least one of volumetric data or pressure data during the procedure.
